(19) United States
(12) Patent Application Publication
(10) Pub. No.: US 2006/0121531 A1
(43) Pub. Date: Jun. 8, 2006

(54) Piezoelectric bio-chip for detecting pathogen of mad cow disease and thereon preparation

(75) Inventors: Chuanzhong Wei, CHONGQING (CN); ZHIHONG Mo, Chongqing (CN); Guiping Ma, Chongqing (CN); Ping Jin, Chongqing (CN); Bingling Li, Chingqing (CN); Xuelong Tian, Chongqing (CN); HAIYAN Tian, Chongqing (CN); Gang Guo, Chongqing (CN)

Correspondence Address: MERCHANT & GOULD PC, P.O. BOX 2903, MINNEAPOLIS, MN, 55402-0903, US

(73) Assignees: BEIJING ENTRY-EXIT INSPECTION AND QUARANTINE, BUREAU OF THE PEOPLE REPUBLIC OF CHINA,, BEIJING (CN); CHONGQING UNIVERSITY, CHONGQING (CN)

(21) Appl. No.: 10/523,174

(22) PCT Filed: May 22, 2003
(86) PCT No.: PCT/CN03/00378

§ 371 Date: Aug. 12, 2005

(30) Foreign Application Priority Data
Jul. 31, 2002 (CN) 02133567.2

(51) Int. Cl. G01N 33/53; C12M 1/34; H01L 21/00

(52) U.S. Cl. 435/7.1; 435/287.2; 435/7.5; 438/1

(57) ABSTRACT

The present invention relates to the device for animal inspection and quarantine and the preparation method thereof. The present invention is especially applicable to the detection of the pathogen of bovine spongiform encephalopathy (BSE) (also known as “mad cow disease”). The present invention employs a piezoelectric chip, a microelectrode array and a common electrode fixed on the lower side surface and upper side surface of the piezoelectric chip, respectively, and a BSE PrP antibody array to constitute the piezoelectric biochip for the detection of the BSE pathogen. The BSE PrP antibodies are immobilized on the electrodes of the microelectrode array in a format corresponding uniquely to the electrodes of the microelectrode array by adsorbing, bonding, cross-linking, embedding or self-assembly process. The combination of the piezoelectric biochip and a detector constitutes the piezoelectric biochip detection system for the BSE pathogen. When the antibodies react with the corresponding PrPs immunochemically, the information about the PrPs can be detected at real time by measuring the resonant frequency, and the PrPs thus can be analyzed qualitatively and quantitatively. The present invention is applicable to the early, effective and rapid detection of the BSE pathogen.

### TECHNICAL FIELD

The present invention relates to the device for animal inspection and quarantine and the preparation method thereof. The present invention is especially applicable to the detection of the pathogen of bovine spongiform encephalopathy (referred to as “BSE” hereinafter).

### BACKGROUND ART

Mad cow disease is a subacute spongiform encephalopathy caused by an unconventional pathogenic agent called prion protein (referred to as “PrP” hereinafter). The BSE pathogen, scrapie-related fibre, is constituted by abnormal PrP resistant to proteases (Prusiner, “Scrapie Prions”, Ann. Rev. Microbiol. 43, 345-374 (1989)). Because PrP has strong resistance against some physical and chemical factors at a degree much higher than various microorganisms and parasites known, and because its strong infectivity and severe damage are extremely harmful to human and animal health, the BSE has caused increasing panic and concern. The detection of the BSE pathogen, PrP, is therefore of great significance.

Proteins are often detected by immunological methods, which comprise preparing the antibody with the test protein as an antigen, and detecting qualitatively or quantitatively or localizing the protein depending on the characteristics in properties and activities of an immunological complex formed by immunochemical reaction of the antigen and the antibody. Likewise, currently available methods for detection of the BSE pathogen, abnormal PrP, are generally based on immunological assays, including immunoelectrophoresis, radioimmunoassay, fluorescent immunoassay, enzyme-linked immunoassay and the like. The abnormal BSE PrP has been detected by fluorescent labeled capillary immunoelectrophoresis and confocal dual-color fluorescent spectroscopy, respectively (Schmerr et al., “Use of Capillary Electrophoresis and Fluorescent Labeled Peptides to Detect the Abnormal Prion Protein in the Blood of Animals that are Infected with a Transmissible Spongiform Encephalopathy”, Journal of Chromatography A, 853, 207-14 (1999); and Bieschke et al., “Ultrasensitive detection of pathological prion protein aggregates by dual-color scanning for intensely fluorescent targets”, Proceedings of the National Academy of Science (USA), 97(10), 5468-73A (2000)). All the detection devices used in these techniques need to employ PrP or PrP antibody labeled with isotopes, fluoresceins, enzymes or the like. Therefore, there have been extremely strict limitations on the detection conditions. Moreover, the detection devices are complex and expensive, and the procedures are laborious and complicated and are difficult to be automatized.

Biochip is a biochemical analysis device developing very rapidly in recent years. For piezoelectric biochip, for example, reference can be made to e.g. the Chinese Patent Application No. 991174402 filed by the Chongqing University to the State Intellectual Property Office of P.R.C. with the title of “Piezoelectric Resonant Micro-Sensor Array Chip” and the Chinese Patent Application No. 001131109 filed by Zhihong Mo et al. with the title of “In Situ Biochip and its Preparation Method”, wherein several piezoelectric biosensors are integrated on a single chip and respective electrodes on the chip are linked to a oscillator circuit, thereby an independent resonance detection unit is formed. By measuring the response signal of each detection unit, e.g. resonant frequency, acoustic impedance spectrum, frequency spectrum, phase and the like, single-dimensional or multi-dimensional information about the composition or the property of the target component or the multi-component system can be obtained, and general, dynamic, real time or in situ characterization of the target can be achieved. However, these known piezoelectric biochips cannot provide the detection information about the BSE pathogen. Up to now, there has been no report about the detection of the BSE pathogen using a piezoelectric biochip.

### SUMMARY OF THE INVENTION

One of the objects of the present invention is to provide a piezoelectric biochip for detection of the BSE pathogen that can determine the information about the BSE pathogen at real time, and the preparation method thereof.

To achieve the object of the present invention, a piezoelectric biochip for the detection of the BSE pathogen has been constructed from a piezoelectric chip, a common electrode which is fixed on the lower side surface of the piezoelectric chip, a microelectrode array which is fixed on the upper side surface of the piezoelectric chip, and an antibody array comprising a plurality of antibodies against BSE PrPs, which antibodies are immobilized on the electrodes of the microelectrode array in a format corresponding uniquely to the electrodes of the microelectrode array.

The piezoelectric biochip for the detection of the BSE pathogen according to the present invention (see FIGS. 1 and 2) comprises a piezoelectric chip comprising a piezoelectric material (1), a common electrode (2), a microelectrode array (3) and a BSE PrP antibody array on the microelectrodes (4). Said piezoelectric chip has a smooth surface and has a common electrode (2) and a microelectrode array consisting of at least two discrete microelectrodes (3) on lower and upper side surfaces of the piezoelectric chip, respectively. The PrP antibodies are antibodies corresponding to any of a variety of PrPs and are immobilized on the electrodes of the microelectrode array in a format corresponding uniquely to the electrodes of the microelectrode array, constituting the PrP antibody array (4) consisting of at least one PrP antibody.

The BSE PrP antibody array (4) above may comprise antibodies against PrPs with various N-terminal amino acid sequences and in either normal or abnormal configurations, thereby making the piezoelectric biochip become a piezoelectric biochip for the detection of the BSE pathogen.

The BSE PrP antibodies can be immobilized on the microelectrodes by adsorbing, bonding, cross-linking, embedding or self-assembly process. Both the binding intensity between the immobilized antibodies and the electrodes and the activity of the immobilized antibodies to react to the test protein are related to factors such as the immobilization method employed, the composition and pH value of the fixing agent used, the immobilization temperature and period of time. Particularly, it has been shown that the immobilized antibodies have strong binding intensity and reactive activity when cross-linking or self-assembly process is used, the pH value of the fixing agent is 4-10, the environmental temperature for the antibody immobilization is in the range of from 0 Celsius degree (° C) to 70° C. and the immobilization period of time is 0.1-24 hours, such that the configuration of the PrP antibodies remains unchanged before and after immobilization.

The BSE PrP antibody array used in the present invention is combined with a piezoelectric resonant array. Particularly, the antibody molecules of the PrP antibody array are immobilized on the microelectrodes of the piezoelectric resonance array in a format corresponding uniquely to the microelectrodes of the piezoelectric resonance array, forming detection sites for individual BSE PrPs. The detection sites for individual PrPs constitute the array for detection of PrPs, the whole of which constitutes the piezoelectric biochip for the detection of the BSE pathogen.

The piezoelectric biochip for the detection of the BSE pathogen according to the present invention is used in combination with a detector. For assembly, the common electrode and the microelectrode array are coupled with the interfaces of the piezoelectric resonance detection circuit of the detector correspondingly, resulting in the piezoelectric biochip detection system. When detecting the BSE pathogen, the sample is loaded onto the chip. Since the resonant frequency of each detection site is inversely proportional to the mass of the material on the surface of the site, the dynamic process of the immunological reaction on each site can be detected at real time or in situ by measuring the resonant frequency of the corresponding detection site when the antibody reacts immunochemically with the BSE PrP, and thereby the corresponding BSE PrP(s) can be analyzed qualitatively and quantitatively.

The present invention has the following advantages and effects over the prior art.

In one aspect, the BSE PrP antibody array used in the present invention can be designed and assembled on the basis of the diagnosis subjects or needs. Particularly, a BSE PrP antibody array may be constructed by combining the antibodies against various PrPs such that this piezoelectric biochip for the detection of the BSE pathogen can detect a plurality of PrPs simultaneously, thereby achieving the object of accurate and rapid detection of the BSE pathogen. The piezoelectric biochip of the present invention has additional advantages such as no need for labeling, simple operation, high specificity and high detection efficiency.

In another aspect, the present invention can be carried out in combination with a detector to measure the resonant frequency. Thus, according to the present invention, real time and in situ detection in high specificity and high precision can be performed simultaneously on all detection sites of the chip. Moreover, the detection device can be simplified and miniaturized.

In still another aspect, the chip of the present invention has simple structure, can be prepared simply and conveniently, is amiable to manufacturing in large scale and is relatively low in cost.

The piezoelectric biochip for the detection of the BSE pathogen according to the present invention in combination with a detector is applicable to the early, efficient and rapid detection of BSE.

### BRIEF DESCRIPTION OF THE DRAWINGS

FIG. 1 is a cut away view of FIG. 2.

FIG. 2 is a top plan view FIG. 1.

FIG. 3 shows the N-terminal amino acid sequences of four PrPs (I-IV) useful in a PrP antibody array according to the present invention.

FIG. 4 is a schematic diagram of a self-assembled antibody according the present invention and its binding with PrP.

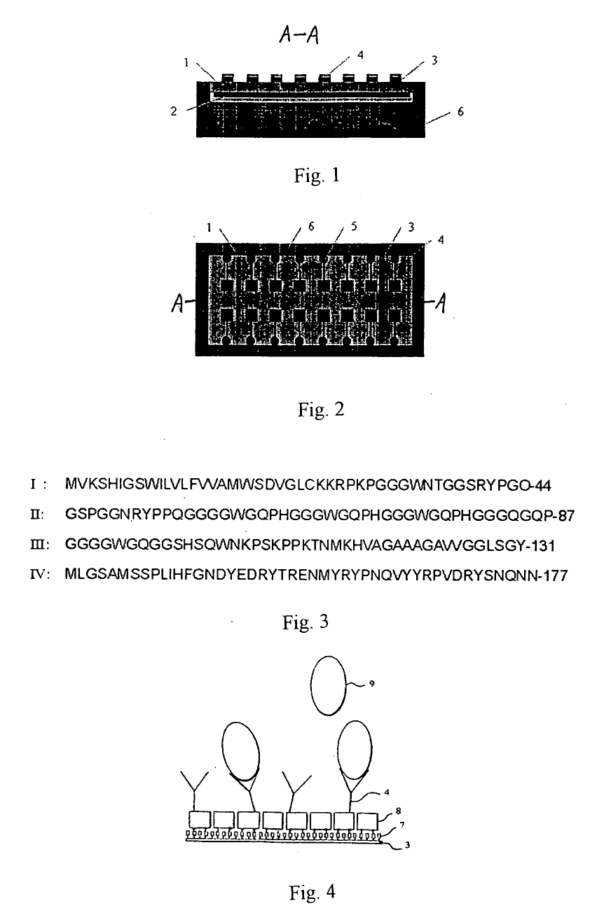

In FIGS. 1 and 2, 1 represents a piezoelectric chip, 2 represents a common electrode, 3 represents a microelectrode array, 4 represents a BSE PrP antibody array, 5 represents an electric wire, and 6 represents a chip support.

In FIG. 3, N-terminal amino acid sequences are set forth according to the criteria of the International Union of Pure and Applied Chemistry (IUPAC). Each letter represents an amino acid as follows: A-alanine, C-cysteine, D-aspartic acid, E-glutamic acid, F-phenylalanine, G-glycine, H-histidine, I-isoleucine, K-lysine, L-leucine, M-methionine, N-asparagine, P-proline, 0-glutamine, R-arginine, S-serine, T-threonine, V-valine, W-tryptophan, and Y-tyrosine.

In FIG. 4, 7 represents a biotin, 8 represents an avidin, and 9 represents a PrP.

Specific Embodiments

The piezoelectric biochip for the detection of the BSE pathogen according to present invention, as shown in FIGS. 1 and 2, is constituted by a piezoelectric chip, a common electrode, a microelectrode array, a BSE PrP antibody array and a chip support.

The piezoelectric chip (1) above may be made of quartz crystal, but other piezoelectric materials such as piezoelectric ceramic or piezoelectric poly(vinylidene fluoride) film may also be used. Piezoelectric chips with smooth surface are produced by conventional methods, and the surface of the chips is in a shape of n-sided polygon with n being equal to or more than 3, such as triangle, quadrangle, pentagon and the like.

The common electrode 2 and the microelectrode array 3 are disposed on the lower side surface and upper side surface of the piezoelectric chip 1, respectively. Commonly used conductive materials such as gold, silver and aluminum may be used. The conductive material can be plated on the surface of both sides of the piezoelectric chip by a vacuum evaporation process to form conductive films. The conductive film on one side of the piezoelectric chip 1 constitutes the common electrode 2. The conductive film on the other side of the piezoelectric chip 1 can be made into microelectrode array 3 consisting of a plurality of discrete, evenly distributed, and patterned microelectrodes, e.g. by photo-engraving or chemical etching processes according to the designed array patterns.

There is no particular limitation on the BSE PrP antibody and the BSE PrP antibody can be any of BSE PrP antibodies known in the prior art, e.g. those described in the references set forth in ‘Background Art’. Moreover, the antibody can be commercially obtained from many suppliers, e.g. those antibodies provided by Prionics (Switzerland) under the Cat. No. 6H4.

The BSE PrP antibody array (4) may comprise antibodies against PrPs with various N-terminal amino acid sequences, and antibodies against PrPs in normal or abnormal configuration. And the BSE PrP antibody array is constituted by PrP antibodies 1-1000 nm thick immobilized on the electrodes of the microelectrode array.

The BSE PrP antibodies may be immobilized on the surface of the electrodes via physical absorption, e.g., by employing the physical properties of Van der Waals' force, electrostatic force and affinity between the electrode metal and the antibody molecule. The PrP antibodies may be immobilized on the surface of the electrodes via chemical bonding, e.g., by the covalent bonding between the reactive free group(s) on a terminal of the antibody molecules, e.g. hydroxyl, carboxyl, or amino, and the corresponding functional group(s) on the pre-treated surface of the electrodes. The PrP antibodies may be immobilized on the surface of the electrodes via cross-linking, e.g. by using a fixing agent with multiple reactive functional groups, e.g. glutaraldehyde, to create cross-linking structure between antibody molecule and the surface of the electrode. The PrP antibodies may be embedded in a porous polymer on the surface of the electrodes. Further, the PrP antibodies may self-assemble themselves on the electrodes by utilizing e.g. the affinity between biotinylated protein and avidin.

The above immobilization methods of the BSE PrP antibodies, particularly, cross-linking and self-assembly, enable a strong binding intensity and reactive activity of the immobilized antibodies by selecting appropriate fixing agent(s), maintaining the immobilization temperature in a range from greater than 0° C. to 70° C. inclusive, and maintaining the pH value of the fixing agent in the range between 4 and 10.

The cross-linking immobilization method of the BSE PrP antibodies may employ aldehyde fixing agents, including formaldehyde, paraformaldehyde, glutaraldehyde and the like; non-aldehyde fixing agents, including carbodiimide, N,N-dimethylacetamide, dimethyloctanoylimide and the like; or the fixing agents as a mixture of aldehyde fixing agent(s) and non-aldehyde fixing agent(s). The fixing agent may also comprise a buffer for the adjustment of the pH value, e.g. commonly used phosphate buffer, acetate buffer and the like.

The immobilization method of the BSE PrP antibodies via self-assembly, as illustrated in FIG. 4, comprises allowing a layer of biotin 7 to be self-assembled on the surface of the electrode 3, then allowing a layer of avidin 8 to be self-assembled on the layer of biotin, and allowing biotinylated PrP antibodies 4 to bind to the layer of avidin, thereby the biotinylated PrP antibodies being immobilized on the surface of the electrode by self-assembly. These biotinylated PrP antibodies 4 thus immobilized are able to capture and bind to the test PrP 9. Since the antibodies on the surface of the electrodes are arranged regularly, this method has good sensitivity and stability.

The support 6 of the chip may be made of ceramic, but other insulative materials such as plastic or glass can also be used. The edge of the piezoelectric chip 1 is fixed upon the edge of the support 6 by thermal compression process or with a binder. The role of the support 6 is to support the piezoelectric chip and it is required that during assembly, both the microelectrodes and the PrP antibodies thereon do not come into contact with the support 6. Thus, the piezoelectric biochip for the detection of the BSE pathogen according to the present invention is constructed.

### EXAMPLES

### Example 1

One piezoelectric biochip for the detection of the BSE pathogen according to the present invention.

In this example, the piezoelectric chip 1 was a chip of quartz crystal which was 100 micrometers (μm) thick, both the microelectrode film 3 and its pin 5 were gold film 200 nanometers (nm) thick, and the piezoelectric chip 1 and the microelectrode 3 were in a shape of quadrangle. The antibody array 4 consisted of four PrP antibodies against normal or abnormal PrPs with N-terminal amino acid sequence identified in I or II, respectively. The support 6 was made of ceramic. The antibodies 4 were immobilized on the microelectrodes 3 by a cross-linking process with a fixing agent, and the thickness of the PrP antibodies was 100-150 nm. The fixing agent consisted of 4% paraformaldehyde, 25% glutaraldehyde, 10% phosphate buffer solution of pH 6-8, and a balance of water. The immobilization temperature was 4° C. and the immobilization period of time was 8 hours. This example of the piezoelectric biochip according to the present invention was useful in detecting qualitatively and analyzing quantitatively all of the BSE pathogens with abnormal PrPs comprising N-terminal amino acid sequences identified in I and II, respectively, at the same time.

### Example 2

One piezoelectric biochip for the detection of the BSE pathogen according to the present invention.

In this example, the piezoelectric chip 1 was a chip of quartz crystal which was 80 micrometers (μm) thick, both the microelectrode film 3 and its pin 5 were silver film 150 nanometers (nm) thick, and the piezoelectric chip 1 and the microelectrode 3 were in a shape of round. The antibody array 4 consisted of six PrP antibodies against normal or abnormal PrPs with N-terminal amino acid sequence identified in I, II, or III, respectively. The support 6 was made of plastic. The antibodies 4 were immobilized on the microelectrodes 3 by a cross-linking process with a fixing agent, and the thickness of the PrP antibodies was 100-150 nm. The fixing agent consisted of 2% ethyl-dimethylaminopropylcarboimide hydrochloride, 25% glutaraldehyde, 10% phosphate buffer solution of pH 6-8, and a balance of water. The immobilization temperature was 15° C. and the immobilization period of time was 4 hours. This example of the piezoelectric biochip according to the present invention was useful in detecting qualitatively and analyzing quantitatively all of the BSE pathogens with abnormal PrPs comprising N-terminal amino acid sequences identified in I, II and III, respectively, at the same time.

### Example 3

One piezoelectric biochip for the detection of the BSE pathogen according to the present invention

In this example, the piezoelectric chip 1 was a piezoelectric poly(vinylidene fluoride) chip which was 200 micrometers (μm) thick, both the microelectrode film 3 and its pin 5 were gold film 100 nanometers (nm) thick, and the piezoelectric chip 1 and the microelectrode 3 were in a shape of quadrangle. The antibody array 4 consisted of eight PrP antibodies against normal or abnormal PrPs with N-terminal amino acid sequence identified in I, II, III, or IV, respectively. The support 6 was made of plastic. The antibodies 4 were immobilized on the microelectrodes 3 by self-assembly of biotin and avidin, and the thickness of the PrP antibodies was 100-500 nm. The immobilization temperature was 25° C. and the immobilization period of time was 2 hours. This example of the piezoelectric biochip according to the present invention was useful in detecting qualitatively and analyzing quantitatively all of the BSE pathogens with abnormal PrPs comprising N-terminal amino acid sequences identified in I, II, III, and IV, respectively, at the same time.

In the above examples, the lower limits of the piezoelectric biochips for the detection of the BSE pathogens were 1-10 ng/mL, and multiple PrPs were be able to be determined simultaneously in ten minutes at a single time.

1. A piezoelectric biochip for the detection of the bovine spongiform encephalopathy (BSE) pathogen, which piezoelectric biochip comprises a piezoelectric chip (1), a common electrode (2) which is fixed on the lower side surface of the piezoelectric chip, and a microelectrode array (3) which is fixed on the upper side surface of the piezoelectric chip, characterized in that a plurality of BSE prion protein (PrP) antibodies are immobilized on the electrodes of the microelectrode array in a format corresponding uniquely to the electrodes of the microelectrode array, thereby forming a BSE PrP antibody array (4).

2. The piezoelectric biochip for the detection of the BSE pathogen according to claim 1, characterized in that in the BSE PrP antibody array (4), PrP antibodies of 1-1000 nm thick are immobilized on the electrodes of the microelectrode array, and that the antibody array comprises antibodies against PrPs with various N-terminal amino acid sequences and in normal and/or abnormal configurations.

3. The piezoelectric biochip for the detection of the BSE pathogen according to claim 2, characterized in that the BSE PrP antibody array consists of antibodies against normal and abnormal PrPs with N-terminal amino acid sequences identified in I and II, respectively, and that the thickness of the PrP antibody is 100-150 nm.

4. A piezoelectric biochip for the detection of the BSE pathogen according to claim 2, characterized in that the BSE PrP antibody array consists of six antibodies against normal and abnormal PrPs with N-terminal amino acid sequences identified in I, II, and III, respectively, and that the thickness of the antibody is 100-150 nm.

5. A piezoelectric biochip for the detection of the BSE pathogen according to claim 2, characterized in that the BSE PrP antibody array consists of eight antibodies against normal and abnormal PrPs with N-terminal amino acid sequences identified in I, II, III, and IV, respectively, and that the thickness of the antibody is 100-500 nm.

6. A method for manufacturing a piezoelectric biochip for the detection of the BSE pathogen, which method comprises:
(1) manufacturing a microelectrode array; and
(2) immobilizing PrP antibodies on the electrodes of the microelectrode array (3) by physical adsorbing, chemical bonding, cross-linking, embedding or self-assembly process, wherein the environmental temperature for antibody immobilization is greater than 0° C. to 70° C. inclusive, and the immobilization period of time is 0.1-24 hours, such that the configuration of the PrP antibodies remains unchanged before and after immobilization.

7. The method for manufacturing a piezoelectric biochip for the detection of the BSE pathogen according to claim 6, characterized in that the antibodies are immobilized on the electrodes of the microelectrode array by a cross-linking process with a fixing agent consisting of 4% paraformaldehyde, 25% glutaraldehyde, 10% phosphate buffer solution at pH 6-8, and a balance of water, wherein the immobilization temperature is 4° C. and the immobilization period of time is 8 hours.

8. The method for manufacturing a piezoelectric biochip for the detection of the BSE pathogen according to claim 6, characterized in that the antibodies are immobilized on the electrodes of the microelectrode array by a cross-linking process with a fixing agent consisting of 2% ethyl-dimethylaminopropyl carboimide hydrochloride, 25% glutaraldehyde, 10% phosphate buffer solution at pH 6-8, and a balance of water, wherein the immobilization temperature is 15° C. and the immobilization period of time is 4 hours.

9. The method for manufacturing a piezoelectric biochip for the detection of the BSE pathogen according to claim 6, characterized in that the antibodies are immobilized on the electrodes of the microelectrode array by self-assembly of biotin and avidin, wherein the immobilization temperature is 25° C. and the immobilization time is 8 hours.
